(19) United States
(12) Patent Application Publication
(10) Pub. No.: US 2008/0281307 A1
(43) Pub. Date: Nov. 13, 2008

(54) THERAPEUTIC DEVICE INCORPORATING LIGHT AND COLD THERAPY MODALITIES

(76) Inventor: Nadine Donahue, Corrales, NM (US)

Correspondence Address: SCHWEITZER CORNMAN GROSS & BONDELL LLP, 292 MADISON AVENUE - 19th FLOOR, NEW YORK, NY, 10017, US

(21) Appl. No.: 11/746,139

(22) Filed: May 9, 2007

(51) Int. Cl. A61B 18/02

(52) U.S. Cl. 606/13

(57) ABSTRACT

A therapeutic device for human and animal use comprises light emitters for applying light energy to a chosen target area of a patient in combination with target area cooling. Control over the intensity, duration and/or sequencing of the light energy is provided, and the light energy therapy may be combined with other modalities. The device may be in the form of a pad, capable of being affixed against the portion of the body, such as a wrist, ankle or neck by a strap.

The present invention relates to an apparatus for the treatment of an animal or human by the application of light energy in association with cold therapy.

### BACKGROUND OF THE INVENTION

The use of light therapy as a treatment modality for animals and humans is well known. For example, U.S. Pat. No. 6,443,978 to Zharov discloses a light therapy device in which the light emitters are mounted on a substrate which conforms to the geometry of the portion of the patient to which the treatment is directed. As recited in the Zharov patent, light treatment may be used in connection with dermatology and cosmetology treatments, as well as for the treatment of trauma, edema, varicose veins, infectious processes and other conditions.

The prior art also teaches the use of light therapy in conjunction with various other energy modalities, such as magnetic field radiation, vacuum therapy, and electrical stimulation. U.S. Pat. No. 6,249,698 to Parris, for example, discloses a treatment device in which different treatment modalities, including light therapy, can be driven and applied simultaneously, modulated at an audio or sub-audio frequency.

While the prior art directed to light therapy teaches the application of other forms of energy to the patient, there are conditions in which the application of multi-modal energy may be inappropriate or ineffective. Further, heating effects associated with the affirmative application of energy to a living organism may cause unwanted or deleterious effects.

It is accordingly a purpose of the present invention to provide a method and apparatus for multi-modal treatment therapies incorporating both light therapy and energy removal, i.e., active cooling of the therapy site.

It is a further purpose of the present invention to provide such multi-modal therapy in an apparatus which is both efficient and convenient in use, and which may be adapted to a variety of treatment sites and therapy plans, including first aid and sports medicine.

### BRIEF DESCRIPTION OF THE INVENTION

In accordance with the foregoing and other objects and purposes, a multi-modal therapy apparatus of the present invention comprises light emitter means mounted to a base or substrate which may be placed in contact with the portion of the patient to be treated. The apparatus further includes means mounted to or otherwise associated with the substrate to provide cooling or cold therapy to the portion of the patient upon which the substrate is placed. The substrate may be, for example, a flexible wrap having a cooling agent suspended or mounted therein, with the light therapy emitters being in the form of one or more light emitting diodes positioned on the surface of the substrate. The diodes may be formed in a matrix to allow a substantial area of the patient's body surface to be treated. Concurrently, the cooling agent extends over an extended area of the substrate, whereby cooling of the entire area to which the light therapy is applied is effected.

The cooling agent not only assists in counteracting heat generated by the light-emitting elements, but further causes a positive cooling or temperature decrease of the patient's body as a treatment modality separate from the application of light.

In addition to light and cooling, additional modalities, such as electrical stimulation, magnetic therapy, etc., may be incorporated into the apparatus. The timing and duration of the application of each such modalities may be controlled by use of appropriate control and timing devices or systems. The apparatus may be in the form of a self-contained system, or may be integrated with other therapeutic devices, such as a neck brace. It may, for example, serve as an integral part of a first aid or EMS system, to apply the modalities to a patient at an accident or other trauma site.

### BRIEF DESCRIPTION OF THE DRAWINGS

A fuller understanding of the present invention will be achieved upon consideration of the following detailed description of preferred but nonetheless illustrative embodiments thereof, when reviewed in conjunction with the annexed drawings, wherein:

FIG. 1 is a side elevation view in section of the invention, intended to be sized and dimensioned to be placed upon the wrist of a patient;

FIG. 2 is a top plan view thereof;

FIG. 3 is a bottom plan view thereof;

FIG. 4 is a top plan view of the invention in the form of an ankle wrap; and

FIG. 5 is a top plan view of the invention in the form of a neck wrap.

### DETAILED DESCRIPTION OF THE INVENTION

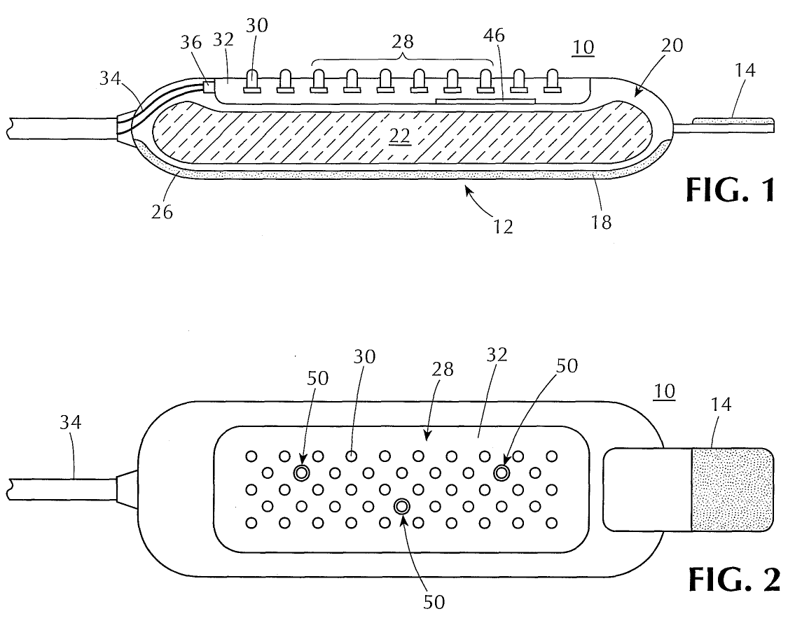

With initial reference to FIGS. 1-3, the present invention may be in the form of a wrap 10, dimensioned and configured to be placed around a portion of the patient's body to which the therapy is to be applied, such as the wrist or other portion of the arm. The wrap comprises a main, therapy-applying portion 12, and attachment means to allow the wrap to be maintained in the proper position on the body. The attachment means may comprise, for example, a strap 14 extending from one end of the therapy-applying portion 12 and a mating element 16 located at the opposite end of the therapy-applying portion. The strap and mating element may be provided with inter-engaging hook and loop fastener elements to secure them together, or other inter-engaging or cinching elements as known in the art.

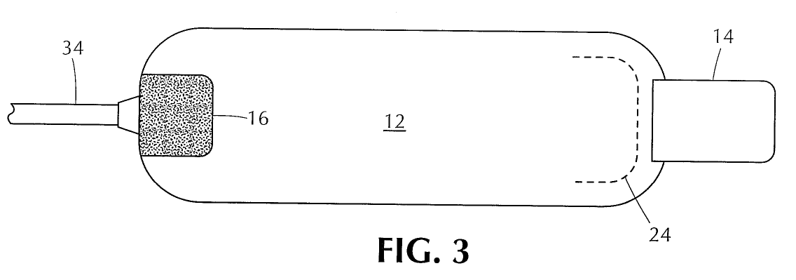

The main therapy-applying portion 12 may be configured as a pad having an outer wall or jacket 18 of a non-allergenic material which may be safely placed in intimate contact with the skin without adverse effects, and may be, for example, medical-grade rubber, fabric or the like. The pad wall 18 creates an inner compartment 20 in which is located cooling element 22. The cooling element 22 may be, for example water, which may be frozen when the wrap 10 is placed in a freezer, the wrap then being removed for use. Alternatively, the cooling element may be a chemical composition which when activated generates an endothermic reaction, drawing heat from the surrounding area, or an electrical cooling device, such as an semiconductor cooling module. The cooling element 22 may be located directly within the inner compartment 20, preferably in a self-contained sub-assembly, particularly when the cooling element is in the form of a liquid or chemical composition. Further, the sub-assembly is in the form of an insertable module or package. In such a case the module or package, such as an ice pack, may be insertable and removable through a resealable or closable slit or opening 24 in the pad jacket 18, as depicted in FIG. 3. Preferably, the cooling element 22 extends substantially completely within the compartment 20, such that its cooling effect migrates outwardly over the entirety of the skin-contacting portion of the pad. In that regard, the construction of the pad may be such as to channel the cooling effects towards the skin. For example, the inner surface of the outwardly-lying side of the therapy-applying portion 12 may be provided with a layer of insulation 26 to lessen the loss of cooling effect directed outwardly and away from the skin and to assist in maximizing the cooling effect applied to the skin.

Located on the skin-facing surface of the therapy-applying portion 12 is a matrix 28 of light-generating elements, such as light-emitting diodes 30. Other emitters, such as fiber optic bundles from one or more light sources, or “micro dot” technology light emitter, can similarly be used. The matrix 28 may be contained in a self-contained electrical assembly 32 positioned within the pad directly adjacent to the skin-directed portion of wall 18, with the diodes or other emitting elements projecting slightly through apertures in the wall. The assembly 32 is preferably packaged to be protected from possible deleterious effects resulting from contact with the cooling element 22. Alternatively, the assembly 32 may be mounted to the exterior surface of the pad wall 18, or may be accommodated in a recess formed therein. A variety of light emitters can be used, within a wide spectral range in the visible, IR and UV spectra as desired. The light energy or flux may likewise be chosen in accordance with intended effects and available technology. Typically, the arrangement of the individual light emitters 30 within the matrix are chosen to provide a generally consistent light output over an area adapted to the geometry of the area to be treated, which can range for small targeted areas to several square inches. The density and nature of the emitters can vary over the therapy area as may be required or desired.

When the light therapy is to be combined with other modalities, the appropriate transducers, as illustrated by magnetic induction coil elements 50, may be incorporated into the matrix assembly.

Using appropriate semiconductor/integrated circuit technology, the matrix assembly 32 may also include an appropriate electronic drive controller 34 for the light matrix and other transducers, if present. The controller may include systems to vary the intensity of the light from individual emitter elements, to switch emitter individual elements on and off in various patterns, to integrate the multiple modalities, if present, and to vary the wavelength of the emitted light in connection with light emitters that may have such capacity. Micro-switches or other programming means (not shown) may be used to program the controller. The matrix assembly 32 may be connected to a series of electrical conductors that extend from the therapy portion 12 as cable 34 that allows the matrix assembly to be powered by a remotely-located power supply (not shown) which can be a battery pack for portable use or a line-powered power pack. While the controller may include a power supply, it is anticipated that a remote power supply would be beneficial, as volume constraints in the pad itself limit the amount of energy storage (e.g. batteries) to be accommodated therein. The remote power supply may further include the functionality of controller 46. Again, it may be advantageous to include at least portions of the controller circuitry with a remote power supply to lessen the size of the matrix assembly 32. It is further contemplated that an interface, represented schematically by connector 36, be provided between the matrix assembly 32 and cable 34. This can facilitate interconnection between the assembly and a variety of control power supply units and can facilitate interchange of matrix assemblies 32 as may be appropriate for desired therapy purposes.

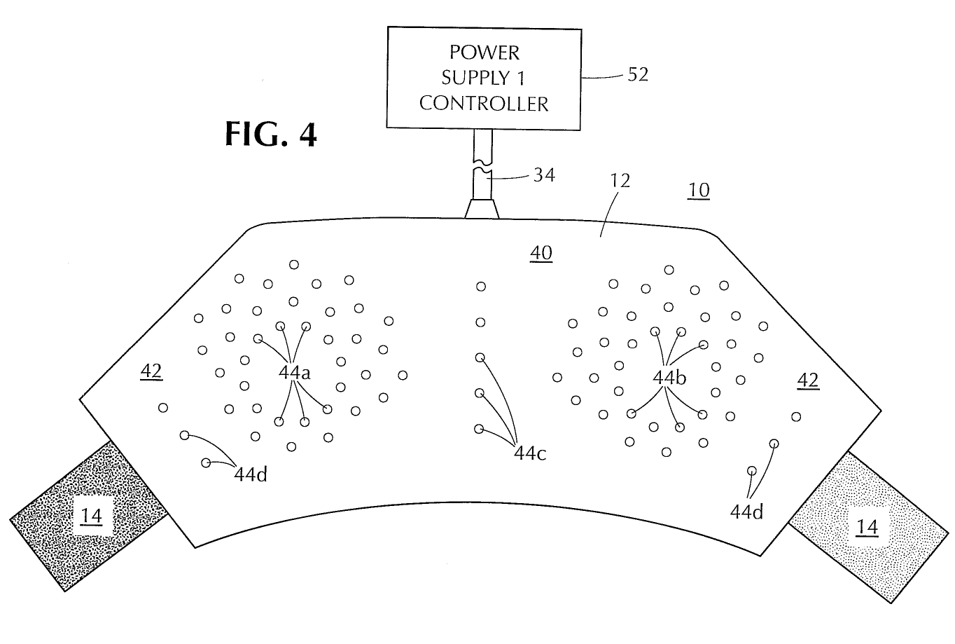

With reference to FIG. 4, an ankle wrap embodiment of the invention is depicted. The therapy-applying portion 12 may be constructed with an elongated central portion 40 bounded by tapered side portions 42. Interconnectable fastening straps 14 extend outwardly from the opposed edges of the side portions. Such a construction allows the wrap to be placed over and about the ankle, the fastening straps 14 interconnecting at the rear of the ankle. The light matrix may be arranged in a plurality of sections, sub-arrays 44a and 44b being located to generally overlie the talus bone and the medial and lateral malleolus, while the centrally-positioned array 44c generally overlies the superior extensor retinaculum. Arrays 44d embrace the Achilles tendon. Cable 34 extends centrally from therapy-applying portion 12. This figure also depicts a remote power supply/controller 52 connected to the cable, which may allow selective operation of each of the sub-arrays.

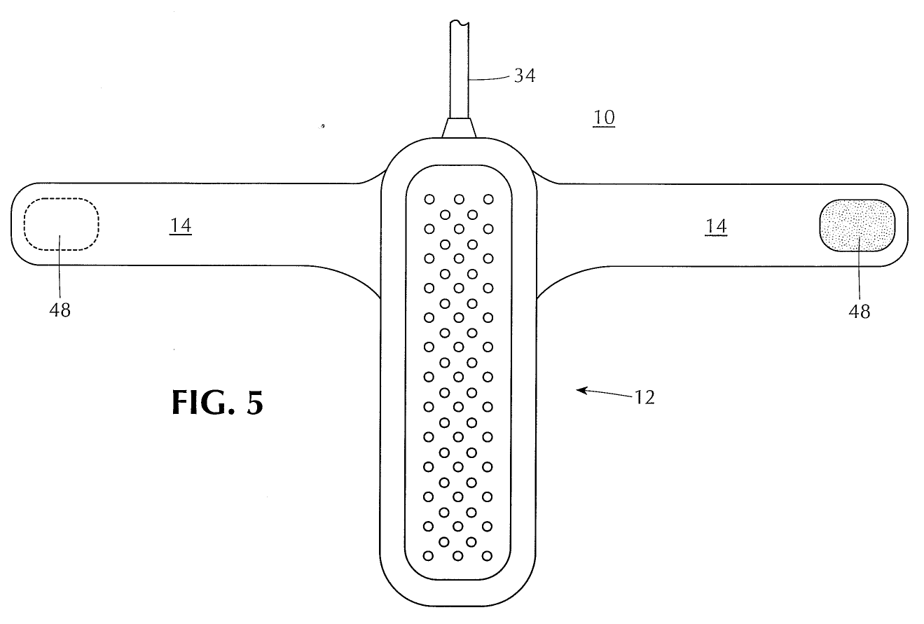

As seen in FIG. 5, the wrap 10 may also be provided in a configuration adapted for the application of therapy to the neck. Therapy-applying portion 12 is in the form of an elongated pad, dimensioned to be placed vertically against the rear of the neck. Fastening straps 14 are located at opposite sides of the portion 12, proximate an end thereof, and are adapted to wrap horizontally around the neck, and may be provided with inter-engagable elements 48, such as complementary hook and loop fastener elements.

I claim:

1. A therapeutic device for human and animal use, comprising a light-emitting means for applying light energy to a chosen target area of a patient, and means for cooling the target area.

2. The device of claim 1, wherein said light-emitting means is coupled to control means for controlling at least one of intensity, duration and sequence of operation of the light-emitting means.

3. The device of claim 2, wherein the light-emitting means and the cooling means are mounted to a substrate.

4. The device of claim 3 further comprising at least one transducer for emitting energy through a non-light modality mounted to the substrate.

5. The device of claim 3 wherein the control means are located at the substrate.

6. The device of claim 3, wherein the substrate is in the form of a pad.

7. The device of claim 6, wherein the pad comprises an outer jacket and an inner chamber, the cooling means being located within the chamber.

8. The device of claim 6, wherein the light-emitting means is in the form of an assembly having a plurality of light-emitting elements, the assembly being positioned against an inner surface of the outer jacket with the light-emitting elements projecting through the outer jacket.

9. The device of claim 6, wherein the cooling means is chosen from an ice pack, a chemical coolant, and an electrical cooling module.

10. The device of claim 6, wherein the cooling means is interchangeable, the outer jacket having means for affording cooling means entry into and removal from the chamber.

11. The device of claim 1 further comprising means for retaining the substrate in contact with the target area.

12. The device of claim 11, wherein the retaining means comprise a strap.

13. The device of claim 3 further comprising a power supply for the light-emitting means located remote from the substrate.

14. The device of claim 13 wherein the control means are located at least partially with the power supply.

15. The device of claim 3 wherein the substrate is a neck pad, wrist wrap or ankle wrap.

16. The device of claim 3 wherein the substrate is an ankle wrap and the light emitting means are formed into at least on sub-array positioned on the substrate to overlie at least one of the talus bone, the medial and lateral malleolus and superior extensor retinaculum.

17. The device of claim 16 wherein the control means includes means for selectively activating the light emitting means sub-arrays.

18. The device of claim 6 wherein the pad includes means for preferentially directing cooling effects of the cooling means towards the target area.

19. The device of claim 18 wherein the directing means comprise a layer of insulation on a portion of the pad directed away from the target area.
